(19) United States
(12) Patent Application Publication
(10) Pub. No.: US 2025/0281662 A1
(43) Pub. Date: Sep. 11, 2025

(54) Motor Vehicle Deodorizer

(71) Applicant: Sensor Electronic Technology, Inc., Columbia, SC (US)

(72) Inventor: Piyush Sharma, Columbia, SC (US)

(21) Appl. No.: 19/067,251

(22) Filed: Feb. 28, 2025

Related U.S. Application Data
(60) Provisional application No. 63/562,757, filed on Mar. 8, 2024.

(51) Int. Cl. A61L 9/20

(52) U.S. Cl. CPC A61L 9/205; A61L 2209/111

(57) ABSTRACT

Deodorization of air, such as for an internal area of a vehicle, using ultraviolet light is described. Ultraviolet light can be emitted from an ultraviolet source and directed onto a surface including a photocatalyst. A processor can control the ultraviolet light emission from the ultraviolet source in response to an indication for deodorization. The deodorization components can be located in a housing which can be configured to be mounted to an internal surface of the vehicle.

### REFERENCE TO RELATED APPLICATIONS

The current application claims the benefit of U.S. Provisional Application No. 63/562,757, filed on 8 Mar. 2024, which is hereby incorporated by reference.

### TECHNICAL FIELD

The disclosure relates generally to deodorization, and more particularly, to a solution for deodorizing one or more areas of a motor vehicle.

### BACKGROUND ART

The motor vehicle industry is continually growing, with more and more vehicles hitting the roads each day. Despite advancements in technology and design, many vehicles still suffer from unpleasant odors that are difficult to remove. A vehicle trunk is an illustrative area from which unpleasant odors can emanate. However, odors also can emanate from the passenger area as well.

Current approaches for eliminating odors from automobile trunks can be ineffective, temporary, or even harmful to human health. Traditional approaches such as using air fresheners, baking soda, or chemical deodorizers may only mask the odor temporarily and may not address the underlying cause of the odor. Moreover, some chemical deodorizers may contain harmful substances that can be detrimental to human health, e.g., causing hazards due to the emission of volatile organic compounds (VOCs).

UV light has been shown to be effective in breaking down a wide range of organic compounds, bacteria, and viruses, and is increasingly being used in a variety of industries and applications for disinfection and deodorization purposes. To this extent, the use of ultraviolet light has been proposed to eliminate unpleasant odors from an automobile trunk. However, to date, no widely utilized or standardized product has been successfully introduced on the market.

Another alternative approach for eliminating odors from vehicle trunks uses ozone generators or ionizers. Ozone generators can be effective in neutralizing odors, but they can also be harmful to human health and can lead to the production of harmful byproducts. Ionizers, on the other hand, may only mask the odor temporarily and may not address the underlying cause of the odor.

### SUMMARY OF THE INVENTION

The inventors propose a solution for removing odors from one or more areas of an automobile which incorporates photocatalytic oxidation (PCO) technology to eliminate the odor at it source. The use of PCO technology for vehicle deodorization can provide a solution that is more effective, safer, and/or longer lasting.

Embodiments of the solution described herein can be deployed in various types of vehicles. In embodiments, a vehicle or vehicle fleet that is utilized by numerous individuals, any one of whom may be sensitive to odors, can utilize embodiments of the solution. Illustrative vehicles and vehicle fleets include taxis, buses, rideshare vehicles, rental cars, limousines, etc.

Aspects of the invention provide a system configured for deodorizing a region of a vehicle including one or more features as defined by the claims. Aspects of the invention provide a method of deodorizing a region of a vehicle using a system configured as described herein. Further aspects of the invention provide for the deodorization of air, such as for an internal area of a vehicle, using ultraviolet light. Ultraviolet light can be emitted from an ultraviolet source and directed onto a surface including a photocatalyst. A processor can control the ultraviolet light emission from the ultraviolet source in response to an indication for deodorization. The deodorization components can be located in a housing which can be configured to be mounted to an internal surface of the vehicle.

A first aspect of the invention provides a vehicle comprising: a vehicle deodorizer component comprising: an ultraviolet source mounted to a first internal surface of the vehicle; a second surface, wherein the ultraviolet light emitted from the ultraviolet source is directed toward the second surface, wherein the second surface includes a photocatalyst; and a processor configured to control emission of ultraviolet light from the ultraviolet source in response to an indication for deodorization of an internal area of the vehicle including the first internal surface.

A second aspect of the invention provides a system for deodorizing air, the system comprising: a housing configured to be mounted to at least one internal surface of a vehicle, the housing including: an ultraviolet source; a surface including a photocatalyst, wherein the ultraviolet light emitted from the ultraviolet source is directed toward the surface including the photocatalyst; and a processor configured to control ultraviolet light emission from the ultraviolet source in response to an indication for deodorization.

A third aspect of the invention provides a vehicle comprising: a vehicle deodorizer component comprising: an ultraviolet source mounted to a first internal surface of the vehicle; a second surface, wherein the ultraviolet light emitted from the ultraviolet source is directed toward the second surface, and wherein the second surface includes a photocatalyst; a sensing device configured to acquire data for an internal area of the vehicle; and a processor configured to control emission of ultraviolet light from the ultraviolet source in response to an indication for deodorization for the internal area of the vehicle, wherein the indication is based on at least one of: an amount of time since a previous deodorization for the internal area, data received by the sensing device, the vehicle being started, the vehicle braking, or the vehicle accelerating.

The illustrative aspects of the invention are designed to solve one or more of the problems herein described and/or one or more other problems not discussed.

### BRIEF DESCRIPTION OF THE DRAWINGS

These and other features of the disclosure will be more readily understood from the following detailed description of the various aspects of the invention taken in conjunction with the accompanying drawings that depict various aspects of the invention.

FIG. 1 shows a schematic diagram of an illustrative vehicle including a vehicle deodorizer according to embodiments.

FIG. 2 shows an illustrative trunk of a vehicle including a vehicle deodorizer according to embodiments.

FIGS. 3A and 3B show an illustrative vehicle deodorizer according to embodiments.

FIG. 4 shows an illustrative flow diagram according to embodiments.

FIG. 5 shows still another illustrative vehicle deodorizer according to embodiments.

It is noted that the drawings may not be to scale. The drawings are intended to depict only typical aspects of the invention, and therefore should not be considered as limiting the scope of the invention. In the drawings, like numbering represents like elements between the drawings.

### DETAILED DESCRIPTION OF THE INVENTION

As indicated above, the inventors propose a solution for removing odors from one or more internal areas of an automobile which incorporates photocatalytic oxidation (PCO) technology to eliminate the odor at its source.

PCO is a process that has been used to purify air and water. A PCO process utilizes the energy of ultraviolet (UV) light to activate a photo-catalyst, such as titanium dioxide (TiO₂), and oxygen in the air to create highly reactive oxygen species, e.g., oxidizing hydroxyl radicals. The reactive oxygen species break down volatile organic compounds (VOCs) and other organic pollutants into harmless substances, such as water (H₂O) and carbon dioxide (CO₂). In embodiments, the invention incorporates a UV light source, such as one or more UV light emitting diodes (LEDs), into a small and portable device, which can provide an effective and efficient solution to eliminate odor in one or more internal areas of vehicles.

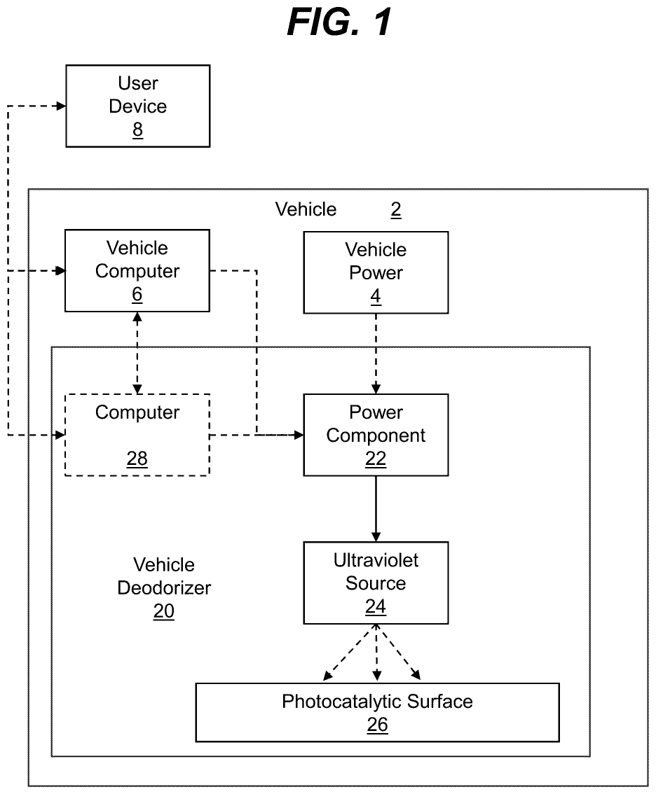

Turning to the drawings, FIG. 1 shows a schematic diagram of an illustrative vehicle 2 including a vehicle deodorizer 20 according to embodiments. As illustrated, a typical vehicle 2 includes a vehicle power component 4, which can deliver power to various locations on the vehicle 2. For example, the power can be delivered to different locations that include a light, a sensor, an electronic control component, and/or the like. Additionally, the vehicle power component 4 can deliver power to one or more interfaces that enable a user device 8 to access and utilize the vehicle power. For example, a console, dashboard, and/or trunk of the vehicle 2 often include one or more power interfaces. With the increasing prevalence of individuals with electronic devices requiring recharging, modern vehicles 2 are often equipped with more power interfaces, including in the back seat.

Furthermore, modern vehicles 2 also can include one or more interfaces that provide both power and connectivity to a vehicle computer 6. The vehicle computer 6 can comprise any type of computer incorporated into the vehicle 2 for operating one or more systems of the vehicle. In embodiments, the vehicle computer 6 can manage operation of an entertainment system, a heating and cooling system, vehicle health monitoring system, and/or the like. For example, a vehicle 2 may include one or more universal serial bus (USB) interfaces, which can enable both data exchange and power delivery between connected devices. Still further, a vehicle 2 also can be configured to enable wireless connectivity between a user device 8 and the vehicle computer 6, e.g., using a short-range wireless communications protocol, such as Bluetooth, via mobile telephone and Internet communications, and/or the like.

In embodiments, the vehicle deodorizer 20 can include a power component 22 which is configured to provide power to one or more ultraviolet sources 24. The power component 22 can include a power source, such as a battery, which can be configured to acquire power from the vehicle power component 4 (e.g., via an existing or added power interface), can be configured to generate or utilize power from a reusable or regenerative power source, such as solar power, wind power, braking, etc.

The ultraviolet source 24 can be configured to generate ultraviolet light that is directed onto a photocatalyst. The ultraviolet source 24 can include one or more of any of various types of ultraviolet light sources. In embodiments, the ultraviolet source 24 includes one or more ultraviolet light emitting diodes. In embodiments, the ultraviolet source 24 can include one or more locations from which ultraviolet is emitted. Such locations can correspond to an emitting surface of an ultraviolet light emitter, such as an ultraviolet light emitting diode, can include a terminal end of a light guiding structure, such as an optical fiber, an emission surface of a housing including one or more ultraviolet light emitters, and/or the like.

The ultraviolet source 24 can be configured such that light emitted by the ultraviolet source 24 is oriented toward and will impinge upon one or more photocatalytic surfaces 26. The photocatalytic surface 26 can comprise any surface which is coated with any type of photocatalyst.

Control of the emission of ultraviolet radiation from the ultraviolet source(s) 24 can be performed via any of various different configurations. For example, the vehicle deodorizer 20 can include a computer 28 which is configured to perform a process for operating the ultraviolet source 24 in order to deodorize an internal area of the vehicle 2. In embodiments, the computer 28 operates the ultraviolet source 24 in response to an indication for deodorization of a corresponding internal area of the vehicle. For example, the indication can correspond to an amount of time since a previous deodorization, in which the computer 28 can be configured to operate the power component 22 to periodically supply power to a set of ultraviolet emitters of the ultraviolet source 24 for a predetermined time at predetermined intervals. Such a configuration can be utilized when there is no risk for a person to be exposed to the ultraviolet light emitted from the ultraviolet source 24 and when the power component 22 has sufficient power to enable such operation for an extended period of time without requiring recharging or replacement of a battery.

In embodiments, the power component 22 can obtain power from one or more regenerative power sources and/or the vehicle power 4. In this case, the indication for deodorization can include the availability of power from the regenerative power source and/or the vehicle power 4. For example, the power component 22 can operate the ultraviolet source 24 in response to the vehicle 2 being started, the vehicle 2 braking, the vehicle 2 accelerating, and/or the like.

In embodiments, the computer 28 can receive and process data from one or more sensors in order to determine whether to turn on or off the ultraviolet source(s) 24. In this case, the computer 28 can be configured to operate the ultraviolet source 24 in response to receiving odor data indicating a presence of a marker or molecule known to be associated with an odor (e.g., from a chemical sensor, a gas sensor, and/or the like), in response to receiving presence data indicating no human or other animal is present in an area to be illuminated (e.g., from a proximity sensor), in response to receiving data indicating the area to be illuminated is enclosed (e.g., from an open or closed sensor for a door, a visible light sensor, etc.), and/or the like. In embodiments, the computer 28 can receive other data which can be used to determine whether deodorization is indicated and/or to adjust one or more aspects of the deodorization. For example, such data can include environmental data, such as a temperature from a temperature sensor, a humidity from a humidity sensor, and/or the like, which the computer 28 can process to determine when deodorization may be desired (e.g., after a period of high temperature and/or humidity).

In embodiments, the vehicle computer 6 can be configured to control operation of the ultraviolet source 24. For example, the vehicle computer 6 can be configured to implement a deodorization process described herein by directly operating the power component 22 to selectively turn on or off the ultraviolet source 24 and/or to instruct the computer 28 to turn on or off the ultraviolet source 24.

In embodiments, a user can receive data regarding operation of the vehicle deodorizer 20 and/or adjust the operation of the vehicle deodorizer 20 via an interface presented by the vehicle computer 6 and/or presented by the user device 8. The user device 8 can retrieve and present data using a browser app or can include another app executing thereon that interfaces with the vehicle deodorizer 20 and/or the vehicle computer 6.

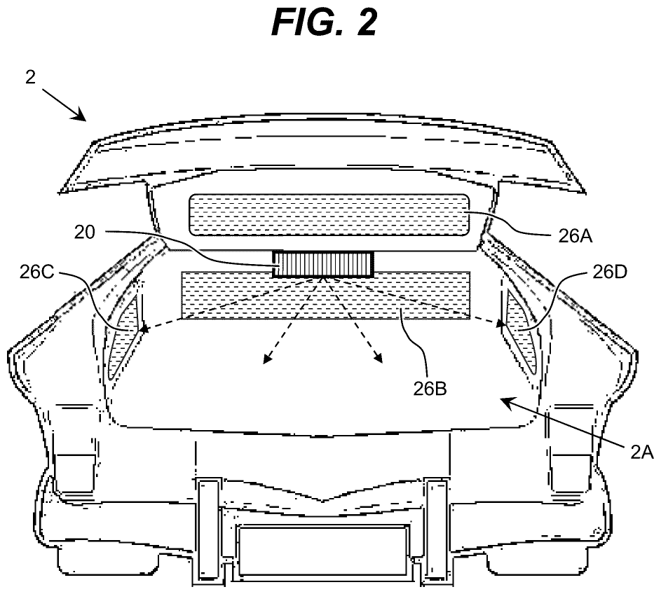

FIG. 2 shows an illustrative trunk 2A of a vehicle 2 including a vehicle deodorizer 20 according to embodiments. In this case, the vehicle deodorizer 20 can be mounted to an interior surface of the trunk 2A and be configured to emit ultraviolet radiation that is directed onto one or more surfaces of the interior of the trunk 2A.

In embodiments, one or more of the interior surfaces of the trunk 2A can comprise a photocatalytic surface, such as photocatalytic surfaces 26A-26D. The surfaces can be selected to reduce a possibility that the photocatalytic surface 26A-26D is obstructed by one or more objects stored in the trunk 2A. For example, a photocatalytic surface 26A can comprise an interior surface of the trunk hatch. The interior surface can be an upper surface or a side surface. Other photocatalytic surfaces 26B-26D are illustrated as being side surfaces. While not shown, it is understood that a floor of the trunk 2A also can include a photocatalytic surface.

As discussed herein, the vehicle deodorizer 20 can obtain power from a separate power source, such as a battery, and/or from the vehicle 2 power system. While the vehicle deodorizer 20 is shown mounted in a particular location, it is understood that it can be mounted anywhere within the vehicle trunk 2A. For example, when the vehicle deodorizer 20 obtains power and/or is operated by the vehicle 2 power system, the vehicle deodorizer 20 can be mounted in a location that allows access to the vehicle power and/or control system, such as near a vehicle power and/or control interface, near another electrical appliance, such as a light, a sensor, a speaker, and/or the like.

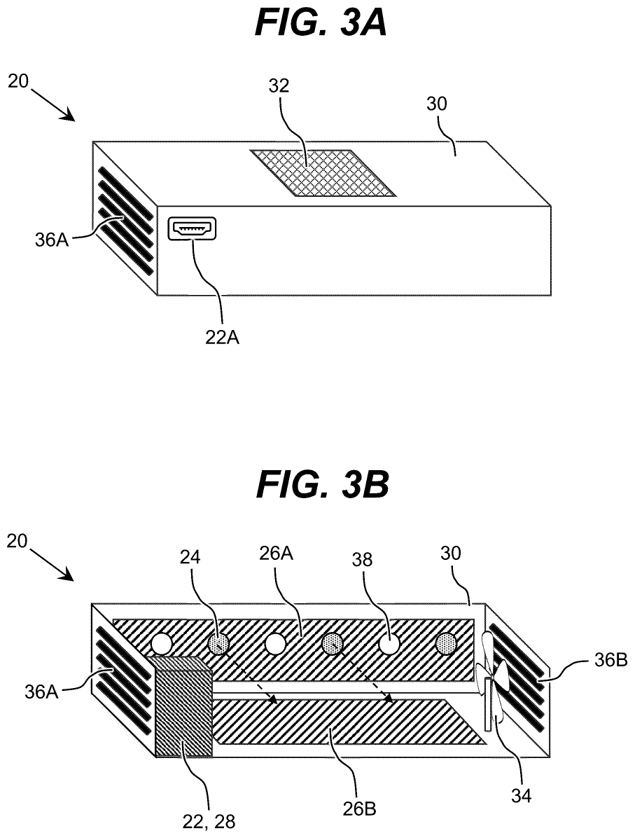

FIGS. 3A and 3B show an illustrative vehicle deodorizer 20 according to embodiments. In this case, the vehicle deodorizer 20 is configured to circulate air through and emit ultraviolet radiation within a housing 30. FIG. 3A shows an outside view of the housing 30 for the vehicle deodorizer 20, while FIG. 3B shows an internal view of the vehicle deodorizer 20 with the top and front sides of the housing 30 removed.

As shown in FIG. 3A, the housing 30 can include a mounting surface 32, which is configured to enable the housing 30 to be mounted to a surface using any solution. In embodiments, the mounting surface 32 can comprise a magnet, which can enable the housing 30 to be mounted to a metal surface. In embodiments the mounting surface 32 can comprise an adhesive surface, which can enable the housing 30 to be mounted to any of various surfaces. Regardless, it is understood that the mounting surface 32 is only illustrative of various solutions that the housing 30 can include for the mounting thereof. To this extent, any permanent or removable/relocatable mounting solution can be used, such as screws, straps, welding, etc.

The housing 30 also is shown including a power and/or control interface 22A. The interface 22A can enable the housing 30 to be connected to a power and/or a control source for operation of the vehicle deodorizer 20. It is understood that the location and type of the interface 22A is only illustrative of various types of interfaces 22A that can be utilized. Additionally, the vehicle deodorizer 20 can use a wireless interface to connect to an externally located control system, such as the vehicle computer, a handheld device, and/or the like. While not shown, the housing 30 also can include one or more human interfaces, such as lights, buttons, a touch screen, and/or the like, which can enable a human to receive information and/or provide operating instructions to the vehicle deodorizer 20.

As illustrated in FIG. 3B, the interior of the housing 30 can include a smaller housing in which a power component 22 and a computer 28 are located for operation of the vehicle deodorizer 20. The power component 22 can include a battery, which can be recharged via the power and/or control interface 22A. Similarly, the computer 28 can operate the ultraviolet source(s) 24 located within the housing 30 according to pre-programmed instructions and/or according to instructions received via the power and/or control interface 22A or another interface described herein. While not shown for clarity, it is understood that the housing 30 can include wiring connecting the power component 22 and the computer 28 to the various components of the vehicle deodorizer 20.

Additionally, the interior of the housing 30 can include a circulation device, such as a fan 34, which the computer 28 can operate to cause air to be circulated within the housing 30. The air can enter and exit the housing 30 through vents 36A, 36B, one or both of which can include a filtration component, such as a HEPA filter or the like. When included, the housing 30 can be configured to enable removal of the filter for replacement and/or cleaning.

Regardless, as illustrated, the air can be circulated such that it passes one or more ultraviolet light sources 24 for treatment. The treatment can include direct irradiation by the light source(s) 24 and/or treatment via photocatalytic oxidation as a result of the ultraviolet light impacting one or more photocatalytic surfaces 26A, 26B. While a particular configuration of the photocatalytic surfaces 26A, 26B is illustrated, it is understood that the interior of the housing 30 can include any configuration of one or more photocatalytic surfaces located in any suitable location. Additionally, the interior of the housing 30 can include reflective surfaces, which can recycle the ultraviolet light emitted by the light source 24. The housing 30 can be configured to prevent the ultraviolet light from escaping the housing 30, e.g., through the use of absorbing surfaces, angled surfaces at the vents 36A, 36B, and/or the like.

In embodiments, the vehicle deodorizer 20 also can include one or more of various types of sensors 38, which can acquire data used by the computer 28 in operating the vehicle deodorizer 20. To this extent, the housing 30 is shown including sensors 38, each of which can acquire data corresponding to one or more attributes of the environment within the interior of the housing 30. Illustrative data can include an air speed, an amount of ultraviolet radiation, a presence or amount of a contaminant, a presence or amount of a reactive oxygen species, etc. Such data can be used by the computer 28 to confirm proper operation of the vehicle deodorizer 20, adjust operation of the vehicle deodorizer 20 based on attributes of the air circulating therein, and/or the like.

As described herein, an embodiment of the vehicle deodorizer 20 can be configured to be permanently or removably mounted to a location in an interior of a vehicle, such as in the trunk of the vehicle. However, it is understood that embodiments of the vehicle deodorizer 20 can be mounted in other locations of the vehicle. For example, embodiments of the vehicle deodorizer 20 can be mounted below a seat in the passenger area of the vehicle, below a dashboard of the vehicle, within a storage space, such as a glove compartment or console, of the vehicle, and/or the like. In further embodiments the vehicle deodorizer 20 can be located within or adjacent to an air circulation component of the vehicle. For example, the vehicle deodorizer 20 can be located near one or more vents through which air enters the passenger area of the vehicle. When mounted within the passenger area of the vehicle, the vehicle deodorizer 20 can be configured to emit ultraviolet radiation contained within a housing and/or directed within a small, isolated area to prevent emission into an area in which a passenger is present.

While the vehicle deodorizer 20 is illustrated as including a housing 30 with various components located therein, it is understood that embodiments of the vehicle deodorizer 20 can be integrated into internal areas of the vehicle without the use of a housing 30. In this case, the ultraviolet source(s) 24, sensor(s) 38, and/or the like, can be mounted directly on or embedded within a surface defining the internal area. For example, the ultraviolet source(s) 24 and/or sensor(s) 38 can be mounted directly to and/or integrated into one or more of the surfaces defining the trunk 2A (FIG. 2), surfaces defining air flow paths within the air circulation system of the vehicle 2, and/or the like.

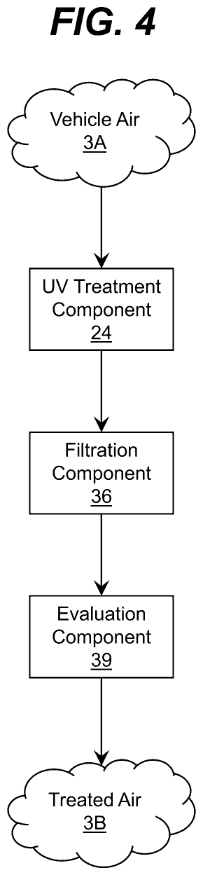

FIG. 4 shows an illustrative flow diagram according to embodiments. In general, during treatment, vehicle air 3A is exposed to ultraviolet light and/or a reactive oxygen species created by the ultraviolet light generated by an ultraviolet treatment component 24. Such exposure can be due to the emission of ultraviolet light within a region of a vehicle, such as the trunk, and/or through circulation of the air past the ultraviolet treatment component 24, which can be housed in a housing. Regardless, the vehicle air 3A also can be filtered by a filtration component 36. While the ultraviolet treatment is shown occurring prior to the filtering, it is understood that this is only illustrative, and the two can occur in any order, including concurrently. Furthermore, while only a single ultraviolet treatment and filtering are shown, it is understood that the vehicle air 3A can undergo multiple ultraviolet treatments and/or filterings, each of which can be configured to target a different organism, molecule, and/or the like.

Embodiments can include an evaluation component 39, which can evaluate the treated air 3B for an effectiveness of the treatment, e.g., using data acquired by one or more sensors 38. In embodiments, the treated air 3B can be recirculated throughout the region of the vehicle and/or within the housing 30, and therefore is reevaluated as vehicle air 3A. The computer 28 can perform the treatment for a predetermined amount of time. In embodiments the computer 28 can stop a treatment when the treated air 3B and/or the vehicle air 3A is indicated as having a sufficiently low level of the target organism(s) and/or compound(s).

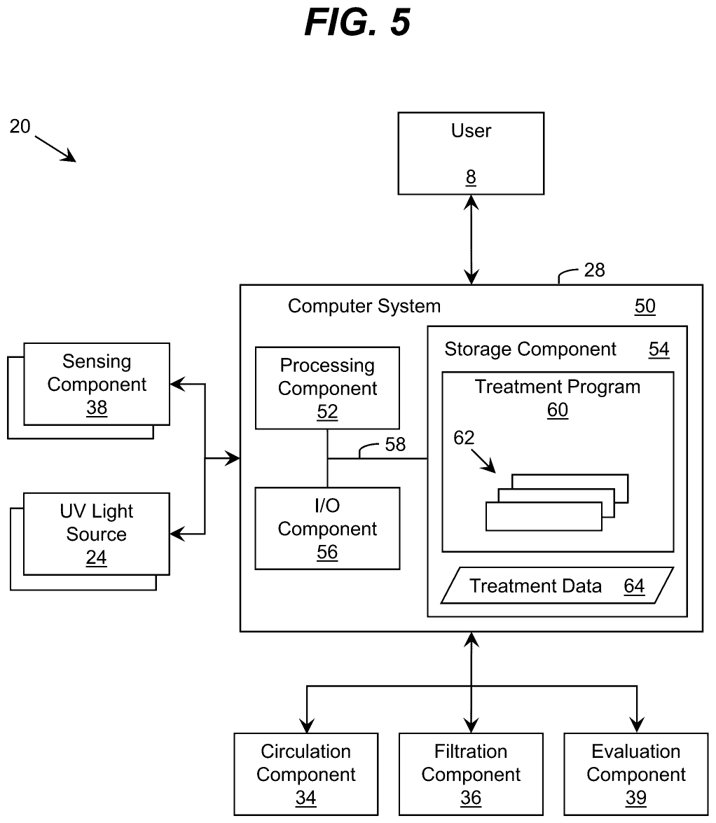

FIG. 5 shows still another illustrative vehicle deodorizer 20 according to embodiments. To this extent, the vehicle deodorizer 20 includes a computing unit 28, which is implemented as a computer system 50 that can perform a process described herein in order to deodorize air within a vehicle. In particular, the computer system 50 is shown including a treatment program 60, which makes the computer system 50 operable to treat the vehicle air by performing a process described herein.

The computer system 50 is schematically illustrated as including a processing component 52 (e.g., one or more processors), a storage component 54 (e.g., a storage hierarchy), an input/output (I/O) component 56 (e.g., one or more I/O interfaces and/or devices), and a communications pathway 58. In general, the processing component 52 executes program code, such as the treatment program 60, which is at least partially fixed in storage component 54. While executing program code, the processing component 52 can process data, which can result in reading and/or writing transformed data from/to the storage component 54 and/or the I/O component 56 for further processing. The pathway 58 provides a communications link between each of the components in the computer system 50.

The I/O component 56 can comprise one or more human I/O devices, which enable a human user 8 to interact with the computer system 50 and/or one or more communications devices to enable a system user 8 (e.g., a portable computing device of a human user, such as a mobile phone executing an app) to communicate with the computer system 50 using any type of communications link. To this extent, the treatment program 60 can manage a set of interfaces (e.g., graphical user interface(s), application program interface, and/or the like) that enable human and/or system users 8 to interact with the treatment program 60 and the treatment data 64. Furthermore, the treatment program 60 can manage (e.g., store, retrieve, create, manipulate, organize, present, etc.) the treatment data 64 using any solution.

In any event, the computer system 50 can comprise one or more general purpose computing articles of manufacture (e.g., computing devices) capable of executing program code, such as the treatment program 60, installed thereon. As used herein, it is understood that “program code” means any collection of instructions, in any language, code or notation, that cause a computing device having an information processing capability to perform a particular action either directly or after any combination of the following: (a) conversion to another language, code or notation; (b) reproduction in a different material form; and/or (c) decompression. To this extent, the treatment program 60 can be embodied as any combination of system software and/or application software.

Furthermore, the treatment program 60 can be implemented using a set of modules 62. In this case, a module 62 can enable the computer system 50 to perform a set of tasks used by the treatment program 60, and can be separately developed and/or implemented apart from other portions of the treatment program 60. As used herein, the term “component” means any configuration of hardware, with or without software, which implements the functionality described in conjunction therewith using any solution, while the term “module” means program code that enables a computer system 50 to implement the actions described in conjunction therewith using any solution. Regardless, it is understood that two or more components, modules, and/or systems may share some/all of their respective hardware and/or software. Furthermore, it is understood that some of the functionality discussed herein may not be implemented or additional functionality may be included as part of the computer system 50.

When the computer system 50 comprises multiple computing devices, each computing device can have only a portion of the treatment program 60 fixed thereon (e.g., one or more modules 62). In embodiments, the computer system 50 can comprise a computing unit associated with the housing 30 and a portable computing unit, such as a mobile phone, which is executing an app installed thereon for enabling a user 8 to monitor, evaluate, manage, and/or the like, the vehicle deodorizer 20. In embodiments, the computer system 50 can comprise a vehicle computer 6 (FIG. 1) as well as a computing unit associated with the housing 30 and/or a portable computing unit, such as a mobile phone.

However, it is understood that the computer system 50 and the treatment program 60 are only representative of various possible equivalent computer systems that may perform a process described herein. To this extent, in other embodiments, the functionality provided by the computer system 50 and the treatment program 60 can be at least partially implemented by one or more computing devices that include any combination of general and/or specific purpose hardware with or without program code. In each embodiment, the hardware and program code, if included, can be created using standard engineering and programming techniques, respectively.

Regardless, when the computer system 50 includes multiple computing devices, the computing devices can communicate over any type of communications link. Furthermore, while performing a process described herein, the computer system 50 can communicate with one or more other computer systems using any type of communications link. In either case, the communications link can comprise any combination of various types of optical fiber, wired, and/or wireless links; comprise any combination of one or more types of networks; and/or utilize any combination of various types of transmission techniques and protocols.

While shown separately from the computer system 50, it is understood that the circulation component 34, filtration component 36, and/or evaluation component 39, can include one or more devices (e.g., sensors, motors, etc.), which provide data processed by the computer system 50 and/or is operated by the computer system 50, in order to perform the corresponding acts.

As used herein, unless otherwise noted, the term “set” means one or more (i.e., at least one) and the phrase “any solution” means any now known or later developed solution. The singular forms “a,” “an,” and “the” include the plural forms as well, unless the context clearly indicates otherwise. Additionally, the terms “comprises,” “includes,” “has,” and related forms of each, when used in this specification, specify the presence of stated features, but do not preclude the presence or addition of one or more other features and/or groups thereof.

As also used herein, a layer is a transparent layer when the layer allows at least ten percent of radiation having a target wavelength, which is radiated at a normal incidence to an interface of the layer, to pass there through. Furthermore, as used herein, a layer is a reflective layer when the layer reflects at least ten percent of radiation having a target wavelength, which is radiated at a normal incidence to an interface of the layer. In an embodiment, the target wavelength of the radiation corresponds to a wavelength of radiation emitted or sensed (e.g., peak wavelength+/−five nanometers) by an active region of an optoelectronic device during operation of the device. For a given layer, the wavelength can be measured in a material of consideration and can depend on a refractive index of the material. Additionally, as used herein, a contact is considered “ohmic” when the contact exhibits close to linear current-voltage behavior over a relevant range of currents/voltages to enable use of a linear dependence to approximate the current-voltage relation through the contact region within the relevant range of currents/voltages to a desired accuracy (e.g., +/−one percent).

It is understood that, unless otherwise specified, each value is approximate and each range of values included herein is inclusive of the end values defining the range. Terms of degree such as “generally,” “substantially,” “about,” and “approximately” as used herein mean a reasonable amount of deviation of the modified term such that the end result is not significantly changed. For example, these terms can be construed as including a deviation of at least +/−0.5% of the modified term if this deviation would not negate the meaning of the word it modifies. In a more particular example, the term “approximately” is inclusive of values within +/−ten percent of the stated value, while the term “substantially” is inclusive of values within +/−five percent of the stated value when these deviations would not negate the meaning of the word each term modifies. Unless otherwise stated, two values are “similar” when the amount of deviation between the two values does not significantly change the result. In a more particular example, two values are similar when the smaller value is within +/−twenty-five percent of the larger value. A value, y, is on the order of a stated value, x, when the value y satisfies the formula 0.1x≤y≤10x.

The foregoing description of various aspects of the invention has been presented for purposes of illustration and description. It is not intended to be exhaustive or to limit the invention to the precise form disclosed, and obviously, many modifications and variations are possible. Such modifications and variations that may be apparent to an individual in the art are included within the scope of the invention as defined by the accompanying claims.

What is claimed is:

1. A vehicle comprising:
a vehicle deodorizer component comprising:
an ultraviolet source mounted to a first internal surface of the vehicle;
a second surface, wherein the ultraviolet light emitted from the ultraviolet source is directed toward the second surface, wherein the second surface includes a photocatalyst; and
a processor configured to control emission of ultraviolet light from the ultraviolet source in response to an indication for deodorization of an internal area of the vehicle including the first internal surface.

2. The vehicle of claim 1, wherein the indication corresponds to at least one of: an amount of time since a previous deodorization for the internal area, data received by at least one sensing device, the vehicle being started, the vehicle braking, or the vehicle accelerating.

3. The vehicle of claim 1, wherein the ultraviolet source is mounted directly to the first internal surface of the vehicle.

4. The vehicle of claim 1, wherein the first internal surface is one of: an internal surface of a trunk, a surface under a seat, a surface below a dashboard, or a surface adjacent to an air vent.

5. The vehicle of claim 1, wherein the second surface comprises a second internal surface of the vehicle.

6. The vehicle of claim 5, wherein the second internal surface is one of: an internal surface of a trunk, a surface under a seat, a surface below a dashboard, or a surface adjacent to an air vent.

7. The vehicle of claim 1, the vehicle deodorizer component further comprising a housing including the ultraviolet source, wherein the second surface comprises a surface of the housing.

8. The vehicle of claim 7, wherein the housing is removably mounted to the internal surface of the vehicle.

9. The vehicle of claim 1, the vehicle deodorizer component further comprising a fan configured to move air over the second surface.

10. The vehicle of claim 1, the vehicle deodorizer component further comprising at least one sensor configured to acquire and provide data for processing by the processor to affect operation of the ultraviolet source.

11. The vehicle of claim 10, wherein the sensor comprises at least one of: a door open or closed sensor, a proximity sensor, a temperature sensor, a humidity sensor, an ultraviolet light sensor, a visible light sensor, a chemical sensor, or a gas sensor.

12. The vehicle of claim 1, wherein the processor comprises at least one of: a vehicle computer configured to manage operation of at least one vehicle system or a portable computing device of a human user.

13. A system for deodorizing air, the system comprising:
a housing configured to be mounted to at least one internal surface of a vehicle, the housing including:
an ultraviolet source;
a surface including a photocatalyst, wherein the ultraviolet light emitted from the ultraviolet source is directed toward the surface including the photocatalyst; and
a processor configured to control ultraviolet light emission from the ultraviolet source in response to an indication for deodorization.

14. The system of claim 13, wherein the housing includes a mounting surface comprising at least one of: a magnetic surface or an adhesive surface.

15. The system of claim 13, wherein the housing is configured for mounting on at least one of: an internal surface of a trunk, a surface under a seat, a surface below a dashboard, or a surface adjacent to an air vent.

16. The system of claim 13, the housing further including a fan configured to move air over the surface including the photocatalyst.

17. The system of claim 13, further comprising at least one sensor configured to acquire data and provide data for processing by the processor to affect operation of the ultraviolet source, wherein the at least one sensor comprises at least one of: a door open or closed sensor, a proximity sensor, a temperature sensor, a humidity sensor, an ultraviolet light sensor, a visible light sensor, a chemical sensor, or a gas sensor.

18. The system of claim 13, wherein the processor receives the indication for deodorization from at least one of: a vehicle computer configured to manage operation of at least one vehicle system or a portable computing device of a human user.

19. A vehicle comprising:
a vehicle deodorizer component comprising:
an ultraviolet source mounted to a first internal surface of the vehicle;
a second surface, wherein the ultraviolet light emitted from the ultraviolet source is directed toward the second surface, and wherein the second surface includes a photocatalyst;
a sensing device configured to acquire data for an internal area of the vehicle; and
a processor configured to control emission of ultraviolet light from the ultraviolet source in response to an indication for deodorization for the internal area of the vehicle, wherein the indication is based on at least one of: an amount of time since a previous deodorization for the internal area, data received by the sensing device, the vehicle being started, the vehicle braking, or the vehicle accelerating.

20. The vehicle of claim 18, wherein the ultraviolet source and the sensing device are located in or on a housing mounted to the first internal surface of the vehicle.
